SEQUENCE LISTING (58 sequences)

SEQ ID NO: 1 - protein, 34 aa, Artificial Sequence
  Insecticidal Protein
XGKICTPAGVKCPAALPCCPGLRCIGGVNNKVCR

SEQ ID NO: 2 - protein, 34 aa, Artificial Sequence
  Insecticidal Protein
GGKICTPAGVKCPAALPCCPGLRCIGGVNNKVCR

SEQ ID NO: 3 - protein, 34 aa, Artificial Sequence
  Insecticidal Protein
AGKICTPAGVKCPAALPCCPGLRCIGGVNNKVCR

SEQ ID NO: 4 - protein, 34 aa, Artificial Sequence
  Insecticidal Protein
SGKICTPAGVKCPAALPCCPGLRCIGGVNNKVCR

SEQ ID NO: 5 - protein, 34 aa, Artificial Sequence
  Insecticidal Protein
VGKICTPAGVKCPAALPCCPGLRCIGGVNNKVCR

SEQ ID NO: 6 - protein, 34 aa, Artificial Sequence
  Insecticidal Protein
TGKICTPAGVKCPAALPCCPGLRCIGGVNNKVCR

SEQ ID NO: 7 - protein, 35 aa, Artificial Sequence
  Insecticidal Protein
GGGKICTPAGVKCPAALPCCPGLRCIGGVNNKVCR

SEQ ID NO: 8 - DNA, 102 bp, Artificial Sequence
  Polynucleotide encoding insecticidal protein
nnnggnaarathtgyacnccngcnggngtnaartgyccngcngcnytnccntgytgyccnggnytnmgntgyathggnggngtnaayaayaargtntgymgn

SEQ ID NO: 9 - DNA, 102 bp, Artificial Sequence
  Polynucleotide encoding insecticidal protein
ggnggnaarathtgyacnccngcnggngtnaartgyccngcngcnytnccntgytgyccnggnytnmgntgyathggnggngtnaayaayaargtntgymgn

SEQ ID NO: 10 - DNA, 102 bp, Artificial Sequence
  Polynucleotide encoding insecticidal protein
ggtggtaarathtgyacnccngcnggngtnaartgyccngcngcnytnccntgytgyccnggnytnmgntgyathggnggngtnaayaayaargtntgymgn

SEQ ID NO: 11 - DNA, 102 bp, Artificial Sequence
  Polynucleotide encoding insecticidal protein
ggcggcaarathtgyacnccngcnggngtnaartgyccngcngcnytnccntgytgyccnggnytnmgntgyathggnggngtnaayaayaargtntgymgn

SEQ ID NO: 12 - DNA, 102 bp, Artificial Sequence
  Polynucleotide encoding insecticidal protein
gcnggnaarathtgyacnccngcnggngtnaartgyccngcngcnytnccntgytgyccnggnytnmgntgyathggnggngtnaayaayaargtntgymgn

SEQ ID NO: 13 - DNA, 102 bp, Artificial Sequence
  Polynucleotide encoding insecticidal protein
wsnggnaarathtgyacnccngcnggngtnaartgyccngcngcnytnccntgytgyccnggnytnmgntgyathggnggngtnaayaayaargtntgymgn

SEQ ID NO: 14 - DNA, 102 bp, Artificial Sequence
  Polynucleotide encoding insecticidal protein
gtnggnaarathtgyacnccngcnggngtnaartgyccngcngcnytnccntgytgyccnggnytnmgntgyathggnggngtnaayaayaargtntgymgn

SEQ ID NO: 15 - DNA, 102 bp, Artificial Sequence
  Polynucleotide encoding insecticidal protein
acnggnaarathtgyacnccngcnggngtnaartgyccngcngcnytnccntgytgyccnggnytnmgntgyathggnggngtnaayaayaargtntgymgn

SEQ ID NO: 16 - DNA, 105 bp, Artificial Sequence
  Polynucleotide encoding insecticidal protein
ggnggnggnaarathtgyacnccngcnggngtnaartgyccngcngcnytnccntgytgyccnggnytnmgntgyathggnggngtnaayaayaargtntgymgn

SEQ ID NO: 17 - DNA, 84 bp, Artificial Sequence
  Polynucleotide encoding signal peptide from Dahlia DmAMP1
atggttaatagatctgttgctttttctgcttttgttcttattctttttgttttggctatttcagatattgcttctgtttcagga

SEQ ID NO: 18 - DNA, 87 bp, Artificial Sequence
  Polynucleotide encoding signal peptide from Radish RsAFP1
atggctaagtttgcttctattattgctcttttgtttgctgcacttgttttgtttgctgcatttgaagctccaactatggttgaagct

SEQ ID NO: 19 - DNA, 72 bp, Artificial Sequence
  Polynucleotide encoding signal peptide from Maize hydroxyproline-rich glycoprotein HRGP
atgggtggcagcggcagggctgctctgctgctggccctggtggccgtgagcctggccgtggagatccaggcc

SEQ ID NO: 20 - DNA, 90 bp, Artificial Sequence
  Polynucleotide encoding signal peptide from Tobacco Pr-1a
atgggatttgttctcttttcacaattgccttcatttcttcttgtctctacacttctcttattcctagtaatatcccactcttgccgtgcc

SEQ ID NO: 21 - DNA, 51 bp, Artificial Sequence
  Polynucleotide encoding signal peptide from Paecilomyces
atgcaaatctccgccgtcattgtcgcactcttcgccagcgccgccatggcc

SEQ ID NO: 22 - protein, 28 aa, Artificial Sequence
  Amino acid sequence of signal peptide from Dahlia DmAMP1
MVNRSVAFSAFVLILFVLAISDIASVSG

SEQ ID NO: 23 - protein, 29 aa, Artificial Sequence
  Amino acid sequence of signal peptide from Radish RsAFP1
MAKFASIIALLFAALVLFAAFEAPTMVEA

SEQ ID NO: 24 - protein, 24 aa, Artificial Sequence
  Amino acid sequence of signal peptide from Maize hydroxyproline-rich glycoprotein (HRGP)
MGGSGRAALLLALVAVSLAVEIQA

SEQ ID NO: 25 - protein, 30 aa, Artificial Sequence
  Amino acid sequence of signal peptide from Tobacco PR-1a
MGFVLFSQLPSFLLVSTLLLFLVISHSCRA

SEQ ID NO: 26 - protein, 17 aa, Artificial Sequence
  Amino acid sequence of signal peptide from Paecilomyces
MQISAVIVALFASAAMA

SEQ ID NO: 27 - protein, 718 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSYHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSLLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 28 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 29 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSYHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYANELHIERNM

SEQ ID NO: 30 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYVGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 31 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVRSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYANELHIERNM

SEQ ID NO: 32 - protein, 719 aa, Bacillus thuringiensis
MKLKNPDKHQSLSSNAKVDKIATDSLKNETDIELKNMNNEDYLRMSEHESIDPFVSASTIQTGIGIAGKILGTLGVPFAGQIASLYSFILGELWPKGKSQWEIFMEHVEEIINQKILTYARNKALSDLRGLGDALAVYHESLESWVENRNNTRARSVVKNQYIALELMFVQKLPSFAVSGEEVPLLPIYAQAANLHLLLLRDASIFGKEWGLSASEISTFYNRQVERTRDYSDHCIKWYNTGLNNLRGTNAKSWVRYNQFRKDMTLMVLDLVALFPSYDTLVYPIKTTSQLTREVYTDAIGTVHPNQAFASTTWYNNNAPSFSAIEAAVIRSPHLLDFLEKVTIYSLLSRWSNTQYMNMWGGHRLESRPIGGALNTSTQGSTNTSINPVTLQFTSRDVYRTESLAGLNLFLTQPVNGVPRVDFHWKFPTLPIASDNFYYLGYAGVGTQLQDSENELPPETTGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTIGFTTPFSFSDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQIHIERNM

SEQ ID NO: 33 - protein, 34 aa, Artificial Sequence
  Insecticidal protein having cysteine residues in specified positions
XXXXCXXXXXXCXXXXXCCXXXXCXXXXXXXXCX

SEQ ID NO: 34 - protein, 34 aa, Artificial Sequence
  Insecticidal protein having cysteine residues in specified positions
XGXXCXXXXXXCXXXXXCCXXXXCXXXXXXXXCX

SEQ ID NO: 35 - protein, 34 aa, Artificial Sequence
  Insecticidal protein having cysteine residues in specified positions
GGXXCXXXXXXCXXXXXCCXXXXCXXXXXXXXCX

SEQ ID NO: 36 - protein, 34 aa, Artificial Sequence
  Insecticidal protein having cysteine residues in specified positions
GGKXCXXXXXXCXXXXXCCXXXXCXXXXXXXXCX

SEQ ID NO: 37 - DNA, 102 bp, Artificial Sequence
  Polynucleotide encoding insecticidal protein
ggggggaarathtgyacnccngcnggngtnaartgyccngcngcnytnccntgytgyccnggnytnmgntgyathggnggngtnaayaayaargtntgymgn

SEQ ID NO: 38 - DNA, 102 bp, Artificial Sequence
  Polynucleotide encoding insecticidal protein
ggaggaaarathtgyacnccngcnggngtnaartgyccngcngcnytnccntgytgyccnggnytnmgntgyathggnggngtnaayaayaargtntgymgn

SEQ ID NO: 39 - protein, 35 aa, Artificial Sequence
  Insecticidal protein
XGKICTPAGVKCPAALPCCPGLRCIGGVNNKVCRX

SEQ ID NO: 40 - protein, 35 aa, Artificial Sequence
  Insecticidal protein
GGKICTPAGVKCPAALPCCPGLRCIGGVNNKVCRX

SEQ ID NO: 41 - protein, 33 aa, Artificial Sequence
  Insecticidal protein (445 or R524445) from Paecilomyces sp. described in WO01/00841
GKICTPAGVKCPAALPCCPGLRCIGGVNNKVCR

SEQ ID NO: 42 - protein, 34 aa, Artificial Sequence
  Insecticidal Protein
RGKICTPAGVKCPAALPCCPGLRCIGGVNNKVCR

SEQ ID NO: 43 - protein, 34 aa, Artificial Sequence
  Insecticidal protein
NGKICTPAGVKCPAALPCCPGLRCIGGVNNKVCR

SEQ ID NO: 44 - protein, 34 aa, Artificial Sequence
  Insecticidal protein
DGKICTPAGVKCPAALPCCPGLRCIGGVNNKVCR

SEQ ID NO: 45 - protein, 34 aa, Artificial Sequence
  Insecticidal protein
CGKICTPAGVKCPAALPCCPGLRCIGGVNNKVCR

SEQ ID NO: 46 - protein, 34 aa, Artificial Sequence
  Insecticidal protein
QGKICTPAGVKCPAALPCCPGLRCIGGVNNKVCR

SEQ ID NO: 47 - protein, 34 aa, Artificial Sequence
  Insecticidal protein
EGKICTPAGVKCPAALPCCPGLRCIGGVNNKVCR

SEQ ID NO: 48 - protein, 34 aa, Artificial Sequence
  Insecticidal protein
HGKICTPAGVKCPAALPCCPGLRCIGGVNNKVCR

SEQ ID NO: 49 - protein, 34 aa, Artificial Sequence
  Insecticidal protein
IGKICTPAGVKCPAALPCCPGLRCIGGVNNKVCR

SEQ ID NO: 50 - protein, 34 aa, Artificial Sequence
  Insecticidal protein
LGKICTPAGVKCPAALPCCPGLRCIGGVNNKVCR

SEQ ID NO: 51 - protein, 34 aa, Artificial Sequence
  Insecticidal protein
KGKICTPAGVKCPAALPCCPGLRCIGGVNNKVCR

SEQ ID NO: 52 - protein, 34 aa, Artificial Sequence
  Insecticidal protein
MGKICTPAGVKCPAALPCCPGLRCIGGVNNKVCR

SEQ ID NO: 53 - protein, 34 aa, Artificial Sequence
  Insecticidal protein
FGKICTPAGVKCPAALPCCPGLRCIGGVNNKVCR

SEQ ID NO: 54 - protein, 34 aa, Artificial Sequence
  Insecticidal protein
PGKICTPAGVKCPAALPCCPGLRCIGGVNNKVCR

SEQ ID NO: 55 - protein, 34 aa, Artificial Sequence
  Insecticidal protein
WGKICTPAGVKCPAALPCCPGLRCIGGVNNKVCR

SEQ ID NO: 56 - protein, 34 aa, Artificial Sequence
  Insecticidal protein
YGKICTPAGVKCPAALPCCPGLRCIGGVNNKVCR

SEQ ID NO: 57 - protein, 16 aa, Impatiens balsamina
SNAADEVATPEDVEPG

SEQ ID NO: 58 - protein, 20 aa, Impatiens balsamina
SNAADEVATPEDVEPGIGKR

(19) United States
(12) Patent Application Publication
(10) Pub. No.: US 2004/0250313 A1
(43) Pub. Date: Dec. 9, 2004

(54) Insecticidal proteins and synergistic combinations thereof

(76) Inventors: Jason Leigh Vincent, Bracknell (GB); Russell Viner, Bracknell (GB)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/478,243

(22) PCT Filed: May 30, 2002
(86) PCT No.: PCT/GB02/02666

(30) Foreign Application Priority Data
Jun. 7, 2001 (GB) 0113900.5

(51) Int. Cl. A01N 63/00; A01H 1/00; C07H 21/04; C12N 15/82; C12N 5/04

(52) U.S. Cl. 800/279; 514/12; 530/324; 536/23.6; 435/419

(57) ABSTRACT

The present invention relates to insecticidal proteins. In a particular embodiment the invention provides an insecticidal protein having the amino acid sequence depicted as SEQ ID No. 1. The invention also provides an insecticidal synergistic protein combination comprising a first insecticidal protein according to the invention in combination with a further protein. Preferably the further protein is an insecticidal crystal endotoxin (CRY) protein Also provided are polynucleotides encoding the proteins and plants which are capable of producing the proteins or protein combination. The proteins according to the invention are particularly suitable for the production of plants which are resistant and/or tolerant to insects.

The present invention relates to inter alia, insecticidal proteins and synergistic combinations thereof, DNA sequences encoding the proteins and methods of producing plants comprising said proteins and combinations. In particular the invention relates to insecticidal peptides which are suitable for expression in plants. The present invention further relates to insecticidal proteins which are capable of acting synergistically with further proteins, in particular CRY and VIP proteins.

According to the present invention there is provided an insecticidal protein comprising an X-glycine (X-G) motif at the N-terminus, wherein X is any amino acid and wherein the insecticidal protein has at least 55% identity with a protein having the sequence XGKICTPAGVKCPAALPCCPGLRCIGGVNNKVCR (SEQ ID No. 1), wherein X is any amino acid.

In a further embodiment of the present invention the insecticidal protein has at least 60% identity to a protein having the sequence of SEQ ID No. 1, wherein X is any amino acid. In a still further embodiment of the present invention the insecticidal protein has at least 65% identity to a protein having the sequence of SEQ ID No. 1, wherein X is any amino acid. In a still further embodiment of the present invention the insecticidal protein has at least 70% identity to a protein having the sequence of SEQ ID No. 1, wherein X is any amino acid. In a still further embodiment of the present invention the insecticidal protein has at least 75% identity to a protein having the sequence of SEQ ID No. 1, wherein X is any amino acid. In a still further embodiment of the present invention the insecticidal protein has at least 80% identity to a protein having the sequence of SEQ ID No. 1, wherein X is any amino acid. In a still further embodiment of the present invention the insecticidal protein has at least 85% identity to a protein having the sequence of SEQ ID No. 1, wherein X is any amino acid. In a still further embodiment of the present invention the insecticidal protein has at least 90% identity to a protein having the sequence of SEQ ID No. 1, wherein X is any amino acid. In a still further embodiment of the present invention the insecticidal protein has at least 91% identity to a protein having the sequence of SEQ ID No. 1, wherein X is any amino acid. In a still further embodiment of the present invention the insecticidal protein has at least 92% identity to a protein having the sequence of SEQ ID No. 1, wherein X is any amino acid. In a still further embodiment of the present invention the insecticidal protein has at least 93% identity to a protein having the sequence of SEQ ID No. 1, wherein X is any amino acid. In a still further embodiment of the present invention the insecticidal protein has at least 94% identity to a protein having the sequence of SEQ ID No. 1, wherein X is any amino acid. In a still further embodiment of the present invention the insecticidal protein has at least 95% identity to a protein having the sequence of SEQ ID No. 1, wherein X is any amino acid. In a still further embodiment of the present invention the insecticidal protein has at least 96% identity to a protein having the sequence of SEQ ID No. 1, wherein X is any amino acid. In a still further embodiment of the present invention the insecticidal protein has at least 97% identity to a protein having the sequence of SEQ ID No. 1, wherein X is any amino acid. In a still further embodiment of the present invention the insecticidal protein has at least 98% identity to a protein having the sequence of SEQ ID No. 1, wherein X is any amino acid. In a still further embodiment of the present invention the insecticidal protein has at least 99% identity to a protein having the sequence of SEQ ID No. 1, wherein X is any amino acid. In a still further embodiment of the present invention there is provided an insecticidal protein which comprises the sequence: XGKICTPAGVKCPAALPCCPGLRCIGGVNNKVCR is (SEQ ID No. 1), wherein X is any amino acid. In a preferred embodiment of the invention X is selected from the group consisting of: Glycine; Alanine; Serine; Valine; Threonine; Cysteine; Asparagine; Glutamine; Phenylalanine and Arginine. In further embodiments, in increasing order of preference, X is Threonine, Serine, Arginine, Phenylalanine, Asparagine, Alanine, or Glycine. Most preferably X is Glycine. In a further embodiment of the present invention said insecticidal protein variant has a Glycine-Glycine-Lysine (G-G-K-) motif at the N-terminus. More preferably the insecticidal protein according to the invention consists of the sequence depicted as SEQ ID No. 2. In a further embodiment of the present invention the insecticidal protein variant consists of a sequence selected from the group depicted as SEQ ID Nos. 3 to 7. In a still further embodiment of the present invention said insecticidal protein variant consists of a sequence selected from the group consisting of SEQ ID Nos. 42 to 56. In a further embodiment of the present invention said insecticidal protein comprises the sequence selected from the group depicted as SEQ ID Nos. 1 to 7, 42 to 56 at the N-terminus. Preferably said insecticidal protein comprises the sequence depicted as SEQ ID No. 2 at the N-terminus.

According to the present invention there is further provided an insecticidal protein consisting of the sequence: XIGKICTPAGVKCPAALPCCPGIRCIGGVNNKVCRXₙ (SEQ ID No. 39) wherein X₁ is any amino acid and n is an integer equal to, or greater than, 1 and when n=1, X is any amino acid and when n>1, each X is independently any amino acid. In a further embodiment of the present invention n is between 1 and 20 inclusive. In a further embodiment of the present invention n is 1. In a still further embodiment of the present invention n is 2. In a still further embodiment of the present invention n is 3. In a still further embodiment of the present invention n is 4. In a still further embodiment of the present invention n is 5. In a still further embodiment of the present invention n is 10. In a still further embodiment of the present invention n is 15. In a still further embodiment of the present invention n is 18. In a still further embodiment of the present invention n is 19. In a still further embodiment of the present invention n is 20. In a still further embodiment of the present invention n>20. In a still further embodiment of the invention X is selected from the group consisting of: Arginine; Lysine and Histidine. In a preferred embodiment of the invention X₁ is selected from the group consisting of: Glycine; Alanine; Serine; Valine Threonine; Cysteine; Asparagine; Glutamine; Phenylalanine and Arginine. In further embodiments, in increasing order of preference, X₁ is Threonine, Serine, Arginine, Phenylalanine, Asparagine, Alanine, or Glycine. Most preferably X₁ is Glycine. The present invention further provides an insecticidal protein variant which has at least 55% identity to SEQ ID No. 39 wherein said variant still has a X₁-Glycine-(X₁-G-) motif at the N-terminus, and X₁ is any amino acid. The present invention further provides an insecticidal protein variant which has at least 65% identity to SEQ ID No. 39 wherein said variant still has a X₁-Glycine-(X₁-G-) motif at the N-terminus, and X₁ is any amino acid. The present invention further provides an insecticidal protein variant which has at least 75% identity to SEQ ID No. 39 wherein said variant still has a X₁-Glycine-(X₁-G-) motif at the N-terminus, and X₁ is any amino acid. The present invention further provides an insecticidal protein variant which has at least 85% identity to SEQ ID No. 39 wherein said variant still has a X₁-Glycine-(X₁G-) motif at the N-terminus, and X₁ is any amino acid. The present invention further provides an insecticidal protein variant which has at least 90% identity to SEQ ID No. 39 wherein said variant still has a X₁-Glycine-(X₁-G-) motif at the N-terminus, and X₁ is any amino acid. The present invention further provides an insecticidal protein variant which has at least 95% identity to SEQ ID No. 39 wherein said variant still has a X₁-Glycine-(X₁-G-) motif at the N-terminus, and X₁ is any amino acid. The present invention further provides an insecticidal protein variant which has at least 96% identity to SEQ ID No. 39 wherein said variant still has a X₁-Glycine-(X₁-G-) motif at the N-terminus, and X₁ is any amino acid. The present invention further provides an insecticidal protein variant which has at least 97% identity to SEQ D No. 39 wherein said variant still has a X₁-Glycine-(X₁-G-) motif at the N-terminus, and X₁ is any amino acid. The present invention further provides an insecticidal protein variant which has at least 98% identity to SEQ ID No. 39 wherein said variant still has a X₁-Glycine-(X₁-G-) motif at the N-terminus, and X₁ is any amino acid. The present invention further provides an insecticidal protein variant which has at least 99% identity to SEQ ID No. 39 wherein said variant still has a X₁-Glycine-(X₁-G-) motif at the N-terminus, and X₁ is any amino acid. In a preferred embodiment of the invention X₁ is selected from the group consisting of: Glycine; Alanine; Serine; Valine; Threonine; Cysteine; Asparagine; Glutamine; Phenylalanine and Arginine. In further embodiments, in increasing order of preference, X₁ is Threonine, Serine, Arginine, Phenylalanine, Asparagine, Alanine, or Glycine. Most preferably X₁ is Glycine. In a further embodiment of the present invention said insecticidal protein variant has a Glycine-Glycine-Lysine (G-G-K-) motif at the N-terminus.

The present invention still further provides an insecticidal protein variant as described above which contains a motif depicted as -LPCCPG- and/or -ICTPA-.

The percentage of sequence identity for proteins according to the invention is determined by comparing two optimally aligned sequences over a comparison window, wherein the portion of the amino acid sequence in the comparison window may comprise additions or deletions (i.e. gaps) as compared to the initial reference sequence (which does not comprise additions or deletions) for optimal alignment of the two sequences. The percentage is calculated by determining the number of positions at which the identical amino acid residue occurs in both sequences to yield the number of match positions, dividing the number of match positions by the total number of positions in the window of comparison and multiplying the result by 100 to yield the percentage of sequence identity. When calculating the percentage sequence identity the sequences may be aligned allowing for up to 3 gaps with the proviso that in respect of the gaps, a total of not more than 15 amino acid residues are affected. Optimal alignment of sequences for comparison may also be conducted by computerised implementations of known algorithms. In a particular embodiment of the present invention the sequence identity is calculated using the FASTA version 3 algorithm which uses the method of Pearson and Lipman (Lipman, D. J. and Pearson, W. R. (1985) Rapid and sensitive protein similarity searches and Science. 227:1435-1441 and Pearson, W. R. and Lipman, D. J. (1988) Improved tools for biological sequence comparison. PNAS. 85:2444-2448) to search for similarities between the reference sequence (also termed the query sequence) and any group of sequences (termed further sequences). Methods also exist in the art which enable the percentage sequence identity between polynucleotide sequences to be calculated.

The protein variant may differ from the basic insecticidal protein sequence (such as SEQ ID No. 1 or 2 for example) by conservative or non-conservative amino acid substitutions. A conservative substitution is to be understood to mean that the amino acid is replaced with an amino acid with broadly similar chemical properties. In particular conservative substitutions may be made between amino acids within the following groups:

(i) Alanine and Glycine;

(ii) Serine and Threonine;

(ii) Glutamic acid and Aspartic acid;

(iii) Arginine and Lysine;

(iv) Asparagine and Glutamine;

(v) Isoleucine and Leucine,

(vi) Valine and Methionine;

(vii) Phenylalanine and Tryptophan.

In general, more conservative than non-conservative substitutions will be possible without destroying the insecticidal properties of the proteins. Suitable variant proteins in accordance with the present invention may be determined by testing insecticidal properties of the protein using routine methods which are well known to the person skilled in the art. Such variant proteins may also be synthesised chemically using standard techniques.

The insecticidal protein or variant according to the invention may also contain at least one additional amino acid at the C-Terminus of the sequence depicted as, or based upon, SEQ ID No. 1.

The present invention still further provides a polynucleotide encoding an insecticidal protein or variant described above.

The present invention still further provides a polynucleotide sequence which is the complement of one which hybridises to a polynucleotide as described above at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still encodes an insecticidal protein having a X-Glycine-(X-G-) motif at the N-terminus of the protein. Preferably, X is an amino acid selected from the group consisting of Glycine; Alanine; Serine; Valine; Threonine; Cysteine; Asparagine; Glutamine; Phenylalanine and Arginine. In further embodiments, in increasing order of preference, X is Threonine, Serine, Arginine, Phenylalanine, Asparagine, Alanine, or Glycine. Most preferably X is Glycine. More preferably said polynucleotide encodes an insecticidal protein having a Glycine-Glycine-Lysine (G-G-K-) motif at the N-terminus. In a further embodiment of the invention the hybridisation is conducted under the following conditions, viz. hybridisation at a temperature of between 60° C. and 65° C. in 0.3 strength citrate buffered saline containing 0.1% SDS followed by rinsing at the same temperature with 0.3 strength citrate buffered saline containing 0.1% SDS.

In a further embodiment of the present invention the polynucleotide sequence comprises the sequence depicted as SEQ ID Nos. 8 to 16, 37 and 38.

Further polynucleotide sequences according to the present invention may be identified from nucleic acid libraries, such as DNA libraries. Suitable oligonucleotide probes may be constructed on the basis of the amino acid sequences of the proteins according to the present invention and used to screen any such library for the identification of further polynucleotides encoding proteins according to the invention. In a still further embodiment of the present invention the amino acid sequence depicted as SEQ ID No. 1 to 7, 42 to 56 may be used for the construction of oligonucleotide probes by the skilled man. In a still further embodiment of the present invention the sequences depicted as SEQ ID Nos. 8 to 16, 37 and 38 may be used for the construction of oligonucleotide probes. The person skilled in the art is well versed in methods for the production and screening of nucleic acid libraries and the necessary techniques for the subsequent identification, isolation and sequence determination of polynucleotides which encode further insecticidal proteins in accordance with the present invention. The person skilled in the art will appreciate that alternative methods exist for the identification and characterisation of related insecticidal sequences from various sources. Such methods include PCR strategies based on oligonucleotide primers using the sequence information provided herein or from sequences obtainable by the methods described above. The person skilled in the art will also appreciate that the polynucleotides according to the invention may also be synthesised ab initio using standard techniques.

In a further aspect of the present invention there is provided an insecticidal synergistic combination consisting of a first protein which is an insecticidal protein as described above and at least one further protein. In a further embodiment of the present invention the further protein is an insecticidal CRY protein. The term “CRY protein” includes crystal endotoxin proteins (and secreted CRY) and the vegetative insecticidal proteins (and secreted VIP) which are active against insects including Lepidoptera, Coleoptera and Diptera. Such proteins are available inter alia, from the bacterium Bacillus thuringienesis and are well known to the person skilled in the art. Particularly preferred CRY proteins which may be used in accordance with the present invention include those proteins obtainable from Bacillus thuringienesis variety tenebrionis which has been deposited under the German Collection of micro-organisms (Deutsche Sammlung von Microorganism) under reference DSM 2803 or strains JHCC 4835 and JHCC 4353 deposited under the National Collections of Industrial and Marine Bacteria (Aberdeen) under the accession numbers NCI 40091 and 40090, respectively. In a still further embodiment of the present invention said further protein comprises a sequence selected from the group consisting of SEQ ID Nos. 27 to 32.

The present invention still further provides a polynucleotide which comprises regions encoding the first and further protein as described above. In a further embodiment of the present invention the polynucleotide comprises a region encoding a first protein which consists of a sequence selected from the group depicted as SEQ ID Nos. 1 to 7, 42 to 56 or a variant protein as described above. Preferably said first protein has a Glycine-Glycine-Lysine (G-G-K-) motif at the N-terminus. In a still further embodiment of the present invention said polynucleotide further comprises a region encoding a sequence selected from the group depicted as SEQ ID Nos. 27 to 32. The insecticidal proteins or protein combinations according to the invention may be prepared in a number of ways which are apparent to the person skilled in the art. For example, by chemical synthesis using a standard peptide synthesiser, or using recombinant DNA technology to express the protein/combination in suitable organisms such as plants and micro-organisms such as E. coli, Saccharomyces cerevisiae or Pichia pastoris.

In a further aspect of the present invention there is provided a method of evolving a polynucleotide which encodes a protein having insecticidal properties comprising: (a) providing a population of variants of said polynucleotide and further polynucleotides which encode further proteins, at least one of which is in cell free form; and (b) shuffling said variants and further polynucleotides to form recombinant polynucleotides; and (c) selecting or screening for recombinant polynucleotides which have evolved towards encoding a protein having the said insecticidal properties; and (d) repeating steps (b) and (c) with the recombinant polynucleotides according to step (c) until an evolved polynucleotide which encodes a protein having insecticidal properties has been acquired wherein said population of variants in part (a) contains at least a polynucleotide as described above. Preferably, said polynucleotide encodes an insecticidal protein having a Glycine-Glycine-Lysine (G-G-K-) motif at the N-terminus. In a further embodiment of the present invention the evolved polynucleotide encodes an insecticidal protein having favourable properties for use in an applied context. For example enhanced activity or efficacy in a particular crop plant.

The present invention still further provides a method as described above wherein said population of variants in part (a) contains at least a polynucleotide encoding the protein depicted as SEQ ID Nos. 1 to 3 and said further polynucleotides in part (a) encode a CRY protein. The present invention still further provides a method as described above wherein said population of variants in part (a) contains at least a polynucleotide encoding the protein depicted as SEQ ID Nos. 4 to 7, 42 to 56 and said further polynucleotides in part (a) encode a CRY protein. The methods for evolving a polynucleotide as described above are well known to the person skilled in the art and are described inter alia, in U.S. Pat. No. 5,811,238.

The present invention still further provides a polynucleotide obtainable or obtained by the methods described above and a protein encoded by any such polynucleotide.

The present invention still further provides a DNA construct comprising in sequence a plant operable promoter operably linked to a polynucleotide encoding a protein as described above operably linked to a transcription termination region. In a further embodiment of the present invention the DNA construct further comprises a region or a plurality of regions which provide for the targeting of the protein product or products to a particular location or locations. For example, if it is desired to provide the protein outside of the cell then an extracellular target sequence may be ligated to the polynucleotide encoding the protein of the present invention. Other examples of targeting include targeting to a specific intracellular organele or compartment such as a chloroplast, any other plastid, endoplasmic reticulum, peroxisome, the oil body, mitochondrion or vacuole. In addition to this, the construct may further comprise a region which provides for an endoplasmic reticulum retention sequence, such as the “KDEL” sequence. Numerous protein targeting sequences are available to the person skilled in the art and any of these sequences may be used to provide either (i) the protein according to the present invention per se and/or (ii) the further protein to, preferably, substantially the same location. In a still further embodiment of the present invention the target sequence comprises a sequence selected from the group depicted as SEQ ID Nos. 17 to 21 or a polynucleotide encoding a protein selected from the group depicted as SEQ ID Nos. 22 to 26. The targeting polynucleotide sequence may be located 5′ and/or 3′ of the polynucleotide encoding the protein or combination according to the present invention.

The present invention still further provides a DNA construct as described above which further comprises a region which provides for the production of a protein which acts as a selectable marker. The selectable marker may, in particular, confer resistance to kanamycin; hygromycin or gentamnycin. Further suitable selectable markers include genes which confer resistance to herbicides such as glyphosate based herbicides or resistance to toxins such as eutypine. Other forms of selection are also available such as hormone based selection systems such as the Multi Auto Transformation (MAT) system of Hiroyrasu Ebinuma et al. 1997. PNAS Vol. 94 pp2117-2121; visual selection systems which use the known green fluorescence protein, β glucoronidase and any other selection system such as mannose isomerase (Positech™), xylose isomerase and 2-deoxyglucose (2-DOG).

The present invention still further provides a DNA construct as described above wherein the plant operable promoter is selected from the group consisting of PolyUbiquitin such as Maize polyubiquitin, Rice pSS1, AoPR1 (such as the promoter obtainable or derivable from Asparagus), Actin2, Agrobacterium rhizogenes RoID; potato protease inhibitor II; CaMV35S; FMV35S; NOS; OCS; Patatin; E9; alcA/alcR switch; GST switch; RMS switch; oleosin; ribulose bisphosphate carboxylase-oxygenase small sub-unit promoter and other root specific promoters including MR7 promoter (maize); Gos 9 (rice) and GOS2 promoters. Terminators which can be used in the constructs according to the present invention include Nos, proteinase inhibitor II and the terminator of a gene of alpha-tubulin (EP-A 652,286). It is equally possible to use, in association with the promoter regulation sequence, other regulation sequences which are situated between the promoter and the sequence encoding the protein according to the present invention, such as transcriptional or translational enhancers, for example, tobacco etch virus (TEV) translation activator described in International Patent application, PCT publication number WO87/07644. The polynucleotide encoding the insecticidal protein or combination according to the invention may also be codon-optimised, or otherwise altered to enhance for example, transcription once it is incorporated into plant material. Examples of preferred codon usage from cotton, maize and rice plants is set out in Table 1 below.

TABLE 1

| | Cotton | Maize | Rice |
|---|---|---|---|
| Amino Acid | preference | preference | Preference |
| Alanine | GCT | GCC | GCC |
| Arginine | AGG | AGG | CGC |
| Asparagine | AAC | AAC | AAC |
| Aspartic Acid | GAT | GAC | GAC |
| Cysteine | TGC | TGC | TGC |
| Glutamine | CAA | CAG | CAG |
| Glutamic Acid | GAG | GAG | GAG |
| Glycine | GGT | GGC | GGC |
| Histidine | CAT | CAC | CAC |
| Isoleucine | ATT | ATC | ATC |
| Leucine | CTT | CTG | CTC |
| Lysine | AAG | AAG | AAG |
| Methionine | ATG | ATG | ATG |
| Phenylalanine | TTC | TTC | TTC |
| Proline | CCT | CCG | CCG |
| Serine | TCT | AGC | TCC |
| Threonine | ACT | ACC | ACC |
| Tryptophan | TGG | TGG | TGG |
| Tyrosine | TAC | TAC | TAC |
| Valine | GTT | GTG | GTG |

Such codon optimisation may also be used to alter the predicted secondary structure of the RNA transcript produced in any transformed cell, or to destroy cryptic RNA instability elements present in the unaltered transcript, thereby increasing the stability and/or availability of the transcript in the transformed cell (Abler and Green. 1996. Plant Molecular Biology (32) pp63-78). The expression of the protein and/or combination according to the present invention may also be enhanced through the inclusion of one or more intronic sequences within the polynucleotide encoding said protein and/or combination. (Rose and Beliakoff, 2000. Plant Physiology (122) pp.535-542). Examples of such sequences are the second intron of the Solanum tuberosum LS 1 gene and the alcohol dehydrogenase 1 gene (adh1) intron of monocotyledonous plant species. The chloroplast expression method (McBride et al. 1995. Biotechnology (13) pp362-365) may also be used to achieve enhanced expression of the protein and/or combination according to the present invention. This method is well known to the person skilled in the art and basically comprises transformation of the chloroplast genome with a polynucleotide under the control of a functional chloroplast-activated promoter or promoter/enhancer combination. The polynucleotide encoding the insecticidal protein according to the invention may also contain other sequence elements such as the so-called Kozak consensus sequences which are well known to the person skilled in the art, for example, cagcc(atg) or agcc(atg).

The proteins and polynucleotides according to the invention are particularly useful in the production of plants which demonstrate levels of resistance and/or tolerance to insects when compared to control-like plants.

In a further aspect of the present invention there is provided a method of providing a plant or plant part with an insecticidal protein or an insecticidal protein synergistic combination comprising: (a) inserting into the genome of plant material a polynucleotide which encodes a protein as described above, or a polynucleotide which is the complement of one which hybridises to a polynucleotide encoding a protein of the invention as described above, or a polynucleotide which comprises regions encoding the first and further protein as described above or a DNA construct as described above; and (b) regenerating plants or plant parts from said material; and (c) selecting the plants or plant parts having said protein or combination. In one embodiment this method provides a plant or plant part with an insecticidal protein synergistic combination by inserting into the genome of plant material that produces a further protein, a polynucleotide which encodes a protein as described above or, a polynucleotide which is the complement of one which hybridises to a polynucleotide encoding a protein of the invention as described above or, a DNA construct as described above. In a still further embodiment there is provided a method of providing a plant or plant part with an insecticidal protein synergistic combination comprising: (a) inserting into the genome of plant material that produces a protein as described above or a protein provided for by a polynucleotide that is the complement of one that hybridises to a polynucleotide encoding a protein as described above, a polynucleotide which provides for a further protein; and (b) regenerating plants or plant parts from said plant material; and (c) selecting the plants or plant parts having said combination. The polynucleotide/DNA construct may be incorporated into the cells by plant transformation techniques that are well known to the person skilled in the art. Such techniques include but are not limited to particle mediated biolistic transformation, Agrobacterium-mediated transformation, protoplast transformation (optionally in the presence of polyethylene glycols); sonication of plant tissues, cells or protoplasts in a medium comprising the polynucleotide or vector; micro-insertion of the polynucleotide or vector into totipotent plant material (optionally employing the known silicon carbide “whiskers” technique), electroporation and the like.

The present invention still further provides a method of providing a plant with an insecticidal protein synergistic combination comprising crossing a first plant which is capable of providing a first protein as described above with a second plant which is capable of producing a further protein and selecting the resultant plant which is capable of producing said combination.

The present invention still further provides plants or plant parts obtained according to the methods as described above.

The present invention still further provides plants or plant parts as described above selected from the group consisting of corn, sweetcorn, melons, mangoes, soybean, cotton, tobacco, sugarbeet, oilseed rape, canola, flax, sunflower, potato, tomato, alfalfa, lettuce, maize, wheat, sorghum, rye, bananas, barley, oat, turf grass, forage grass, sugar cane, pea, field bean, rice, pine, poplar, apple, peaches, grape, strawberries, carrot, lettuce, cabbage, onion, citrus, cereal, nut plants, and other horticultural crops. In a preferred embodiment said plants or plant parts are rice, cotton and corn. Plants and plant parts in accordance with the present invention show improved resistance or enhanced tolerance to an insect pest when compared to control-like or wild-type plants. Resistance may vary from a slight increase in tolerance to the pest to total resistance so that the plant is unaffected by the presence of pest (where the pest is severely inhibited or killed).

The present invention still further provides a method of providing a plant or plant part with a further desired agronomic trait comprising: (a) inserting into the genome of plant material a polynucleotide which provides for the desired agronomic trait; and (b) regenerating plants or plant parts from said material; and (c) selecting the plants or plant parts having said desired agronomic trait wherein said plant material is capable of producing an insecticidal protein or an insecticidal protein combination as described above; or crossing a first plant which plant is capable of producing an insecticidal protein or an insecticidal protein combination as described above with a second plant which provides for said further desired agronomic trait and selecting the resultant plant which is capable of producing the further agronomic trait. In a further embodiment of the present invention the said further desired agronomic trait is selected from the group consisting of: herbicide resistance; insect resistance; nematode resistance; stress tolerance; altered yield; altered nutritional value or any other desirable agronomic trait. In a further embodiment of the present invention the further agronomic trait provides resistance to a herbicide which comprises glyphosate acid or agriculturally acceptable salt thereof.

The present invention still further provides plants or plant parts obtained according to the method of the preceding paragraph.

In a further aspect of the present invention there is provided an insecticidal protein consisting of the sequence depicted as:

| | |
|---|---|
| Xaa₁-Xaa₂-Xaa₃-Xaa₄-Cys₅-Xaa₆-Xaa₇-Xaa₈-Xaa₉-Xaa₁₀-Xaa₁₁-Cys₁₂- | (SEQ ID No. 33) |
| Xaa₁₃-Xaa₁₄-Xaa₁₅-Xaa₁₆-Xaa₁₇-Cys₁₈-Cys₁₉-Xaa₂₀-Xaa₂₁-Xaa₂₂-Xaa₂₃- | |
| Cys₂₄-Xaa₂₅-Xaa₂₆-Xaa₂₇-Xaa₂₈-Xaa₂₉-Xaa₃₀-Xaa₃₁-Xaa₃₂-Cys₃₃-Xaa₃₄- | |

wherein X1-4, 6-11, 13-17, 20-23, 25-32 and 34 are any amino acid. Preferably Xaa₁ is selected from the group consisting of: Glycine; Alanine; Serine; Valine; Threonine; Cysteine; Asparagine; Glutamine; Phenylalanine and Arginine. More preferably Xaa₂ is Glycine (SEQ ID No. 34). Even more preferably Xaa₁ and Xaa₂ are Glycine (SEQ ID No. 35). Even more preferably Xaa₁ and Xaa₂ are Glycine and Xaa₃ is Lysine (SEQ ID No. 36). In a further embodiment of the present invention the insecticidal protein comprises a sequence selected from the group depicted as SEQ ID Nos. 33 to 36 at the N-terminus. In the present case, the insecticidal peptides depicted as inter alia, SEQ ID Nos. 1 to 7, 33-36, 39, 40, 42 to 56 and the proteins encoded by SEQ ID Nos. 8 to 16, 37 and 38 contain six cysteine residues all of which are believed to be involved in forming 3 intramolecular disulphide bonds. Thus the arrangement of the cysteine residues may be important in conferring insecticidal activity on the peptide. In a still further embodiment of the present invention the N-terminal region of the insecticidal protein comprises the sequence GGKICT-.

The present invention still further provides a method of controlling insects comprising providing at a locus where the insects feed, a protein or a protein combination as described above.

The present invention still further provides the use of a polynucleotide encoding an insecticidal protein as described above or a DNA construct as described above in a method for the production of plants or plant parts which are resistant to insects. In a still further embodiment of the present invention the polynucleotide comprises the sequence selected from the group depicted as SEQ ID Nos. 8 to 16.

The present invention still further provides the use of a protein or a protein combination as described above as an active ingredient of a pesticide.

The present invention still further provides a recombinant micro-organism which provides for production of a protein or a protein combination as described above. In a further embodiment of the present invention the microorganism is an endophyte. An endophyte is generally accepted within the art as a micro-organism having the ability to enter into non-pathogenic endosymbiotic relationships with a plant host. A method of endophyte-enhanced protection of plants has been described in a series of patent applications by Crop Genetics International Corporation (for example, International Application Publication Number WO90/13224, European Patent Publication Number EP 125468B 1, International Application Publication Number WO91/10363, International Application Publication Number WO87/03303). International Patent Application Publication Number WO94/16076 (ZENECA Limited) describes the use of endophytes which have been genetically modified to express a plant-derived insecticidal peptide.

The present invention still further provides a recombinant baculovirus which comprises a protein or a protein combination as described above. The present invention still further provides the use of a baculovirus according to the preceding sentence in a method of controlling insects.

According to a further aspect of the present invention there is provided an insecticidal protein which is capable of reacting with a monoclonal antibody raised to the protein depicted as SEQ ID No. 1. The present invention further provides a protein which is capable of reacting with a monoclonal antibody raised to the protein depicted as SEQ ID No. 2. The present invention further provides a protein which is capable of reacting with a monoclonal antibody raised to the protein depicted as SEQ ID No. 3. The present invention further provides a protein which is capable of reacting with a monoclonal antibody raised to the protein depicted as SEQ ID No. 4. The present invention further provides a protein which is capable of reacting with a monoclonal antibody raised to the protein depicted as SEQ ID No. 5. The present invention further provides a protein which is capable of reacting with a monoclonal antibody raised to the protein depicted as SEQ ID No. 6. The present invention further provides a protein which is capable of reacting with a monoclonal antibody raised to the protein depicted as SEQ ID No 7. The present invention still further provides an insecticidal protein which is capable of reacting with a monoclonal antibody raised to a protein selected from the group depicted as SEQ ID Nos. 42 to 56. The present invention still further provides an insecticidal protein which is capable of reacting with a polyclonal antibody raised to a protein selected from the group depicted as SEQ ID No. 1 to 7, 42 to 56. Such antibodies may be generated and used to identify other proteins within the ambit of the present invention according to well-known techniques within the art.

The present invention still further provides a composition comprising an insecticidally effective amount of a protein or a protein combination as described above and optionally an agriculturally acceptable carrier and/or a diluent and/or an insect attractant The composition may be applied to the insects or to the environment in which they live, in particular, to plant parts or the surrounding soil, using standard agricultural techniques for example spraying. The insecticidal proteins and combinations according to the present invention may also be combined in application with other agrochemicals such as herbicides, fungicides and other insecticidal compounds including other insecticidal proteins. Examples of possible mixture partners include insecticidal lectins, insecticidal protease inhibitors and insecticidal proteins derived from species of the Bacillus thurigiensis, Xenorhadus nematophilus, or Photorabdus luminescens and other chemicals for example pyrethroids, carbamates, imidacloprid, organochlorines, macromolecules such as spinosad abamectin or emamectin.

The present invention still further provides a polynucleotide having a first region encoding a protein as described above and a second region encoding a further protein. The regions may be separated by a region which provides for a self processing polypeptide which is capable of separating the proteins such as the self processing polypeptide described in U.S. Pat. No. 5,846,767 or any similarly functioning element. Alternatively the protein regions may be separated by a sequence such as the Ib-IP4 from Impatiens balsamina (SNAADEVATPEDVEPG) or the IP4-Kex2 Linker which uses the Ib-IP4 from Impatiens balsamina with Kex2 protease recognition site (SNAADEVATPEDVEPGIGKR). Such sequences act as a target site for an external element which is capable of separating the protein sequences. Alternatively the polynucleotide may provide for a polyprotein which comprises a plurality of protein functions. In a further embodiment of the present invention the proteins of the polyprotein may be arranged in tandem. In a still further embodiment of the present invention the polyprotein comprises a plurality of protein functions which are separated by linker sequences. Such polyproteins may comprise the proteins and/or further proteins according to the present invention and optionally further proteins such as those encoding any desired argronomic trait.

The present invention still further provides a plant cell comprising a protein or protein combination as described above or a polynucleotide encoding an insecticidal protein and/or an insecticidal protein combination as described above.

The present invention still further provides an insecticidal protein comprising the motif depicted as -LPCCPG- and/or -ICTPA- wherein said protein also has an X-G-motif at the N-terminus where X is any amino acid. Preferably X is selected from the group consisting of: Glycine; Alanine; Serine; Valine; Threonine; Cysteine; Asparagine; Glutamine; Phenylalanine and Arginine. In further embodiments, in increasing order of preference, X is Threonine, Serine, Arginine, Phenylalanine, Asparagine, Alanine, or Glycine and is most preferably Glycine. Even more preferably said protein has a G-G-K-motif at the N-terminus.

The insects to be controlled by the proteins of the present invention include the plant chewing insects and the plant chewing stages of insects such as insect larvae including: Coleoptera, Lepidoptera, Orthoptera and Drosophila, including, but not limited to: Acanthoscelides obtectus, Bruchus sps., Callosobruchus sps. (bruchid beetles), Agriotes sps. (wireworms), Amphimallon sps. (chafer beetles), Anthonomus grandis (cotton boll weevil), Ceutorhynchus assimilis (cabbage seed weevil), Cylas sps. (sweet potato weevils), Diabrotica sps. (corn root worms), Epicauta sps. (black blister beetles), Epilachlia sps. (melon beetles etc.), Leptinotarsa decemlineata (Colorado potato beetle) Meligisthes sps. (blossom beetles), Melolontha sps. (cockchafers), Phyleotreta sps., Psylliodes sps. (flea beetles), Popillia japonica (Japanese beetle), Scolytus sps. (bark beetles), Sitophilus sps. (grain weevils), Tenebrio molitor (yellow mealworm), Tribolium sps. (flour beetles), Trogoderma granarium (Khapra beetle), Acleris sps. (fruit tree tortrixs), Acraea acerata (sweet potato butterfly), Agrotis sps. (cutworms), Autographa gamma (silver-Y moth), Chilo sps. (stalk borers), Cydia pomonella (codling moth), Diparopsis sps. (red bollworms), Ephestia sps. (warehouse moths), Heliothis sps., Helicoverpa sps. (budworms, bollworms), Mamestra brassicae (cabbage moth), Manduca sps. (hornworms), Maruca testulalis (mung moth), Mythimna sps. (cereal armyworms), Ostrinia nubilalis (European corn borer), Pectinophora gossypiella (pink bollworm), Phthorimaea operculella (potato tuber moth), Pieris brassicae (large white butterfly), Pieris rapae (small white butterfly), Plodia interpunctella (Indian grain moth), Plutella xylostella (diamond-back moth), Sitatroga cerealella (Angoumois grain moth), Spodoptera sps. (armyworms), Trichoplusia ni (cabbage semilooper), Acheta sps. (field crickets), Gryllotalph sps. (mole crickets), Locusta migratoria (migratory locust), Schistocerca gregaria (desert locust), Acrythosiphon pisum and Drosophila sp.

The invention will now be described by way of the following non-limiting examples in combination with the following figures and sequence listing of which:

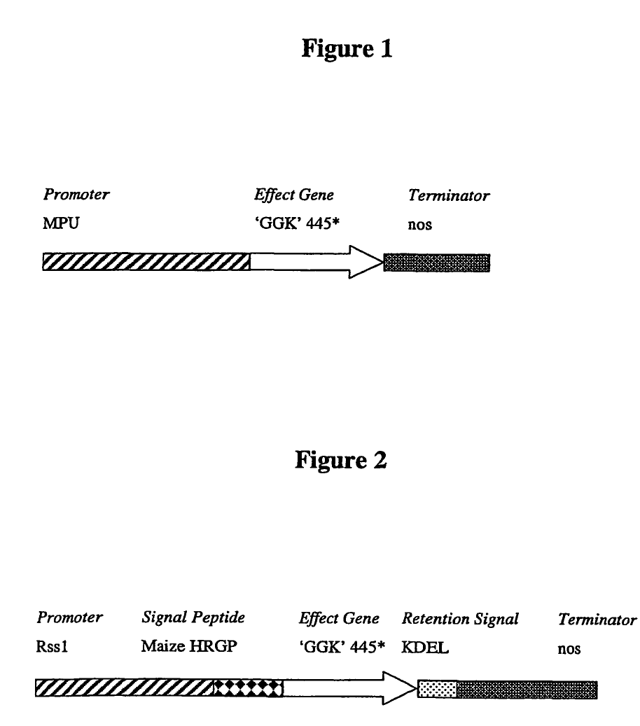

FIG. 1—Maize polyUbiquitin+intron (NPU) promoter drives constitutive expression of the target gene in the plant. In this case, the peptide, SEQ ID No. 2 (also referred to as “GGK 445”) would be ported to the cytoplasm of the cell. KEY: * indicates that the natural coding sequence has been modified, in accordance with the degeneracy of the genetic code, for the purpose of codon optimisation in a target monocot species such as Oryza sativa, Rice.

FIG. 2—Rice Sucrose Synthase (RSS1) promoter directs phloem-preferred expression, so that there is no expression in the grain. Alternatively, the Phosphoenol Pyruvate Carboxylase (PepC) promoter could be used for green tissue expression. The maize Hydroxyproine-Rich Glycoprotein signal peptide targets the protein to the secretory pathway, where it is cleaved by peptidase activity. The KDEL signal at the C-terminus results in retention of the peptide in the endoplasmic reticulum, leading to accumulation of peptide inside the cell. KEY: * indicates that the natural coding sequence has been modified, in accordance with the degeneracy of the genetic code, for the purpose of codon optimisation in a target monocot species such as Oryza sativa, Rice.

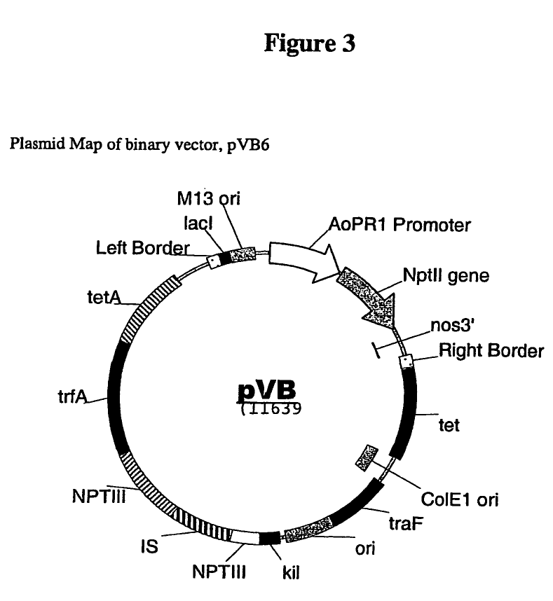

FIG. 3—Plasmid map of binary vector pVB6.

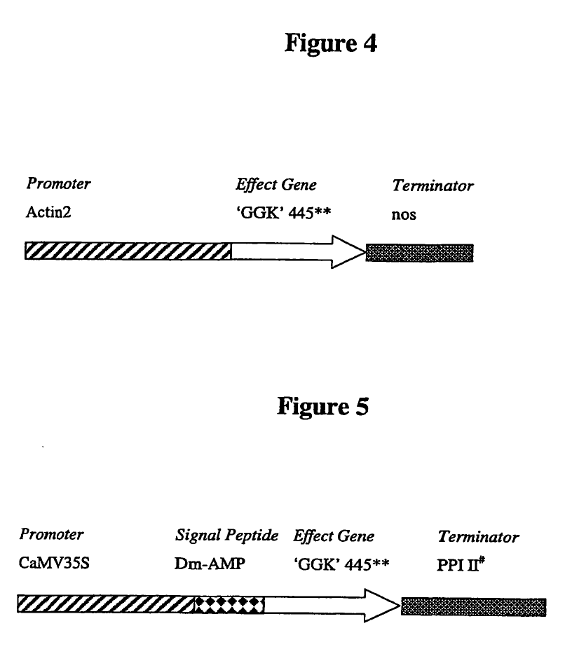

FIG. 4—Construct suitable for expression in dicotyledonous crops comprises the Actin2 promoter which drives constitutive expression of the target gene in the plant. In this case, the peptide, “GGK-445” would be retained in the cytoplasm of the cell. KEY: ** indicates that the natural coding sequence has been modified, in accordance with the degeneracy of the genetic code, for the purpose of codon optimisation in a target dicot species such as Gossypium hirsutum, Cotton.

FIG. 5—Construct suitable for expression in dicotyledonous crops comprises the Cauliflower mosaic virus (CaMV35S) constitutive promoter resulting in expression throughout the plant. The Cotton Rubisco or Ubi3 constitutive promoters may be used if preferred. The Dahlia (Dm-AMP) signal peptide targets the protein to the secretory pathway, where it is cleaved by peptidase activity. The peptide is secreted to the apoplast, outside of the cell. KEY: ** indicates that the natural coding sequence has been modified in accordance with the degeneracy of the genetic code, for the purpose of codon optimisation in a target dicot species such as Gossypium hirsutum, Cotton; # indicates PPI II potato protease inhibitor II terminator.

### DESCRIPTION OF SEQUENCE LISTING

SEQ ID Nos. 1-7=Insecticidal proteins.

SEQ ID Nos. 8-16=Polynucleotides encoding insecticidal proteins.

SEQ ID Nos. 17-21=Polynucleotide sequences encoding the signal peptides from Dahlia (DnAMP1), Radish (RsAFP1), Maize (hydroxyproline-rich glycoproten (HRGP)), Tobacco (PR-1a signal) and Paecilomyces respectively.

SEQ ID Nos. 22 to 26—Amino acid sequences of the signal peptides from Dahlia (Dm-AMP-1), Radish (Rs-AFP1), Maize (hydroxyproline-rich glycoproten (HRGP)), Tobacco (PR-1a signal) and Paecilomyces respectively.

SEQ ID Nos. 27-32=Protein sequences for insecticidal proteins cry1Ia1 (Embl. Accession No. X62821); cry1Ia2 (Embl. Accession No. M98544); cry1Ia3 (Embl. Accession No. L36338); cry1Ia4 (Embl. Accession No. L49391); cry1Ia5 (Embl. Accession No. Y08920) and cry1Ib1 (Embl. Accession No. U07642) respectively.

SEQ ID No. 33-36=Insecticidal protein sequences having cysteine residues in specified positions.

SEQ ID Nos. 37-38=Polynucleotides encoding insecticidal proteins.

SEQ ID Nos. 39-40=Insecticidal proteins.

SEQ ID No. 41=Insecticidal protein (445 or R524445) from Paecilomyces sp. described in WO01/00841.

SEQ ID Nos. 42 to 56=Insecticidal proteins.

### EXAMPLES

### Example 1

Peptide Synthesis

Various proteins based on the sequence information as above were synthesised chemically using standard techniques well known to the person skilled in the art. These proteins include the sequences depicted as SEQ ID Nos. 1 to 7 and 42 to 56 as described in the sequence listing.

### Example 2

Comparitive Insect Bioassays

These were carried out between the synthetic peptide having the sequence depicted as SEQ ID No. 2 (also referred to as the GGK-445 peptide) and the insecticidal peptide obtainable from the fungus Paecilomyces farinosus (referred to as “445”) and described in International Patent Application Publication Number WO01/00841. This protein, having the sequence depicted as SEQ ID No. 41, was synthesised, purified and the N-terminal Glycine was acetylated.

Prior to the assay, twenty neonate Heliothis virescens larvae were gently brushed into each of three ‘minipot’ containers per treatment (i.e. three replicates per treatment). The peptide to be tested was diluted using sterile, deionised water to create a range of test concentrations for determination of a kill curve.

Three freshly excised cotton leaves per treatment had 0.05 ml of the appropriate peptide solution applied by pipette to the centre of the axial surface of each leaf. The droplet was then spread over a circular area in excess of the diameter of a minipot with a fine artists paint brush (a fresh paint brush being used for each compound to avoid contamination), the concentrations being applied from lowest to highest. The leaves were left in a fume cupboard just long enough for the surface deposit to dry, but care was taken to avoid excessive leaf wilting.

Once dry the leaves were placed, contaminated surface down over the appropriately labelled, pre-infested minipot and a lid snapped over it. The minipots were placed in plastic trays and placed in a controlled environment room at 25-27° C.

After three days the numbers of live larvae remaining were counted and percent mortality determined. Percent mortality for the peptide treatments was corrected in comparison with the water-treated control using the Abbott's formula. Damage to the leaf was also assessed and represented as percent feeding inhibition. The results of the comparison are shown in Table 2 below.

TABLE 2

| Trt. | | Dose | corrected | % feeding reduction |
|---|---|---|---|---|
| No. | Treatment | (ppm) | % kill | compared to control |
| 1 | Water-treated control 199998/P1 | 0 | — | — |
| 2 | M576-GGK-445 | 1000 | 94.4 | 81.3 |
| 3 | M576-GGK-445 | 333.33 | 51.9 | 62.5 |
| 4 | M576-GGK-445 | 111.11 | 25.9 | 50.1 |
| 5 | M576-GGK-445 | 37.04 | 11.1 | 25.1 |
| 6 | M576-GGK-445 | 12.35 | 7.4 | 6.4 |
| 7 | M576-GGK-445 | 4.12 | 0.0 | 6.4 |
| 8 | M576-GGK-445 | 1.37 | 0.0 | 0.0 |
| 9 | M576-GGK-445 | 0.46 | 0.0 | 0.0 |
| 10 | M576-GGK-445 | 0.15 | 0.0 | 0.0 |
| 11 | Y1945F - synthesised, acetylated-445 | 1000 | 100.0 | 91.3 |
| 12 | Y1945F - synthesised, acetylated-445 | 333.33 | 98.1 | 88.8 |
| 13 | Y1945F - synthesised, acetylated-445 | 111.11 | 98.1 | 83.8 |
| 14 | Y1945F - synthesised, acetylated-445 | 37.04 | 72.2 | 68.8 |
| 15 | Y1945F - synthesised, acetylated-445 | 12.35 | 75.9 | 68.8 |
| 16 | Y1945F - synthesised, acetylated-445 | 4.12 | 48.1 | 43.8 |
| 17 | Y1945F - synthesised, acetylated-445 | 1.37 | 13.0 | 0.0 |
| 18 | Y1945F - synthesised, acetylated-445 | 0.46 | 5.6 | 0.0 |
| 19 | Y1945F - synthesised, acetylated-445 | 0.15 | 0.0 | 0.0 |

### Example 3

LC₅₀ Determination for Different Insect Pests.

Mean LC₅₀ values were determined for the synthetic peptide, GGK-445, having the amino acid sequence designated in SEQ ID No. 2. The method for testing insect mortality is the same as described in Example 2. The test data was run through a logit analysis package to establish the LC₅₀ values for each test. Table 3 shows individual test and mean LC₅₀ data

TABLE 3

| | LC50 (ppm) | | | |
|---|---|---|---|---|
| | Heliothis | Helicoverpa | Spodoptera | Spodoptera |
| Sample | virescens | armigera | exigua | littoralis |
| GGK-445 | 49.67 | 113.24 | 339.21 | 235.16 |
| (Y1993 12M) | 30.23 | 421.93 | 972.5 | 626.72 |
| | 171.15 | 857.29 | 271.32 | 371.71 |
| | | 1236.39 | | 201.91 |
| | | | | 283.26 |
| means | 83.68 | 657.21 | 527.68 | 343.75 |

### Example 4

Expression of the Insecticidal Peptides in Monocotyledenous Plants.

4.1 A monocot crop such as rice may be transformed so as to express an insecticidal protein according to the invention such as the protein depicted as SEQ ID No. 2 using methods that are well known to the person skilled in the art. Examples of constructs suitable for such monocot expression are summarised in FIGS. 1 and 2. For example, FIG. 1 shows the Maize polyUbiquitin+intron (MPU) promoter which provides for constitutive expression of the target gene in the plant. In this case, the peptide depicted as SEQ ID No. 2 would be retained in the cytoplasm of the cell.

4.2 FIG. 2 shows the use of the Rice Sucrose Synthase (RSS1) promoter which provides for phloem-preferred expression, so that there is no expression in the grain. Alternatively, the Phosphoenol Pyruvate Carboxylase (PepC) promoter could be used for green tissue expression. The maize Hydroxyproline-Rich Glycoprotein signal peptide targets the protein to the secretory pathway, where it is cleaved by peptidase activity. The KDEL signal at the C-terminus results in retention of the peptide in the endoplasmic reticulum, leading to accumulation of peptide inside the cell.

4.3 These gene cassettes may be cloned into a suitable binary vector background, such as pVB6 (FIG. 3) containing a selectable marker gene. These constructs may be used to produce transgenic plants using transformation methods well known to the person skilled in the art. Regenerated transformed plant tissue may be subjected to a bioassay to determine insecticidal activity. Resultant recombinant plants will be tolerant and/or resistant to insects when compared to control-like and/or wild-type plants.

### Example 5

5.1 Expression of the Insecticidal Peptides in Dicotyledenous Plants.

A dicotyledonous crop such as cotton may be transformed so as to express the insecticidal protein according to the invention. Examples of suitable constructs designed for such expression are summarised in FIGS. 4 and 5. FIG. 4 shows the Actin2 promoter which provides for constitutive expression of the target gene in the plant. In this case, the peptide depicted as SEQ ID No.2 would be retained in the cytoplasm of the cell.

5.2 FIG. 5 shows the Cauliflower mosaic virus (CaMV35S) which provides for expression throughout the plant. Alternatively, the Cotton Rubisco or Ubi3 promoters could be used. The Dahlia (Dm-AMP) signal peptide targets the protein to the secretory pathway, where it is cleaved by peptidase activity. The peptide is secreted to the apoplast, outside of the cell.

5.3 These gene cassettes may be cloned into a suitable binary vector background via restriction digestion/ligation. A suitable vector would be pVB6 (FIG. 3), which contains a selectable marker gene such as Hygromycin. These constructs can be used to transform suitable dicotyledenous plants using transformation methods known to the skilled man. Resultant recombinant plants will be tolerant and/or resistant to insects when compared to control-like and/or wild-type plants.

### Example 6

Insecticidal Activity of the Protein Combination

6.1 Previously prepared European Corn Borer (ECB) artificial diet was dispensed in small quantities into tubes and held in a warm water bath at approximately 70° C. An equal amount of diet was added to each tube, and then an equal fixed volume of the appropriate test sample was added. The test samples comprised a mixture of the cry1Ia1 protein (SEQ ID No.27) and the protein depicted as SEQ ID No. 2. The “incorporated diet” was mixed well and approximately 180 ml aliquots were pipetted into petri dishes, giving five replicates for each sample.

6.2 The dishes were infested for between 1-5 hours after the diet is dispensed with five 1^{st} instar larvae per dish/rep and then lidded. The test was held in the dark at approximately 27° C. and 70-80% RH and the insects were assessed five days after treatment for mortality. The results indicate synergistic levels of activity when the protein according to the invention and the cry1Ia1 protein are combined.

1. An insecticidal protein comprising an X-glycine motif at the N-terminus, wherein X is any amino acid and wherein the insecticidal protein has at least 55% identity with a protein having the sequence XGKICTPAGVKCPAALPCCPGLRCIGGVNNKVCR (SEQ ID No. 1), wherein X is any amino acid.

2. The insecticidal protein according to claim 1, which has at least 90% identity with a protein having the sequence: XGKICTPAGVKCPAALPCCPGLRCIGGVNNKVCR (SEQ ID No. 1), wherein X is any amino acid.

3. The insecticidal protein according to claim 1, which comprises the sequence: XGKICTPAGVKCPAALPCCPGLRCIGGVNNKVCR (SEQ ID No. 1), wherein X is any amino acid.

4. The insecticidal protein according to claim 1 wherein X is selected from the group consisting of: Glycine; Alanine; Serine; Valine; Threonine; Cysteine; Asparagine; Glutamine; Phenylalanine and Arginine.

5. The insecticidal protein according to claim 4 consisting of the sequence:

| | |
|---|---|
| GGKICTPAGVKCPAALPCCPGLRCIGGVNNKVCR. | (SEQ ID No. 2) |

6. An insecticidal protein having the sequence: X₁GKICTPAGVKCPAALPCCPGLRCIGGVNNKVCRXₙ (SEQ ID No. 3), wherein X₁ is any amino acid and n is an integer equal to, or greater than, 1 and when n=1, X is any amino acid and when n>1, each X is independently any amino acid.

7. A polynucleotide encoding a protein according to claim 1.

8. A polynucleotide sequence which is the complement of one which hybridises to the polynucleotide according to claim 7 at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still encodes an insecticidal protein having a X-Glycine-(X-G-) motif at the N-terminus wherein X is any amino acid.

9. The polynucleotide sequence according to claim 7 comprising a sequence selected from the group depicted as SEQ ID Nos. 8 to 16, 37 and 38.

10. An insecticidal synergistic combination comprising a first protein according claim 1 and at least one further insecticidal protein.

11. The combination according to claim 10 wherein said further insecticidal protein is a CRY protein.

12. The combination according to claim 11 wherein the said further insecticidal protein comprises a sequence selected from the group consisting of SEQ ID Nos. 27 to 32.

13. A polynucleotide which comprises regions encoding the first and further insecticidal protein according to claim 10.

14. The polynucleotide according to claim 13 wherein the region encoding said first protein comprises a sequence selected from the group depicted as SEQ ID Nos. 8 to 16, 37 and 38.

15. A method of evolving a polynucleotide which encodes a protein having insecticidal properties comprising:
(a) providing a population of variants of said polynucleotide and further polynucleotides which encode further proteins, where at least one of said polynucleotides is in cell free form, and
(b) shuffling said variants and further polynucleotides to form recombinant polynucleotides; and
(c) selecting or screening for recombinant polynucleotides which have evolved towards encoding a protein having the said insecticidal properties; and
(d) repeating steps (b) and (c) with the recombinant polynucleotides according to step (c) until an evolved polynucleotide which encodes a protein having insecticidal properties has been acquired wherein said population of variants in part (a) contains at least a polynucleotide according to claim 7.

16. The method according to claim 15 wherein said population of variants in part (a) contains at least a polynucleotide encoding a protein selected from the group depicted as SEQ ID Nos. 1 to 3 and/or said further polynucleotides in part (a) encode a CRY protein.

17. A polynucleotide obtainable or obtained by the method according to claim 15.

18. A protein encoded by the polynucleotide according to claim 17.

19. A DNA construct comprising in sequence a plant operable promoter operably linked to the polynucleotide according to claim 7 operably linked to a transcription termination region.

20. The DNA construct according to claim 19 which further comprises a region which provides for the targeting of the protein product to a particular location.

21. The DNA construct according to claim 19 which further comprises a region which provides for the production of a protein which acts as a selectable marker.

22. The DNA construct according to claim 19 wherein the plant operable promoter is selected from the group consisting of: PolyUbiquitin, Maize polyubiquitin, Rice pSS1, AoPR1, Actin2, Agrobacterium rhizogenes RolD; potato protease inhibitor II; CaMV35S; FMV35S; NOS; OCS; Patatin; E9; alcA/alcR switch; GST switch; RMS switch; oleosin; ribulose bisphosphate carboxylase-oxygenase small sub-unit promoter and other root specific promoters including MR7 promoter (maize); Gos 9 (rice), and GOS2 promoters.

23. A method of providing a plant or plant part with an insecticidal protein comprising:
(a) inserting into the genome of plant material a polynucleotide according a DNA construct according to claim 19; and
(b) regenerating plants or plant parts from said plant material; and
(c) selecting the plants or plant parts having said protein.

24. A method of providing a plant or plant part with an insecticidal protein synergistic combination comprising:
(a) inserting into the genome of plant material which produces an insecticidal protein, a DNA construct according to claim 19; and
(b) regenerating plants or plant parts form said plant material; and
(c) selecting the plants or plant parts having said combination.

25. A method of providing a plant or plant part with an insecticidal protein synergistic combination comprising:
(a) inserting into the genome of plant material that comprises a polynucleotide according to claim 7, a polynucleotide which provides for a further insecticidal protein; and
(b) regenerating plants or plant parts from said plant material; and
(b) selecting the plants or plant parts having said combination.

26. A method of providing a plant with an insecticidal synergistic combination, comprising crossing a first plant which comprises a polynucleotide according to claim 7 with a second plant which is capable of producing a further insecticidal protein and selecting the resultant plant which is capable of producing said combination.

27. Plants or plant parts obtained according to the method of claim 23.

28. Plants or plant parts according to claim 27 selected from the group consisting of: corn, sweetcorn, melons, mangoes, soybean, cotton, tobacco, sugarbeet, oilseed rape, canola, flax, sunflower, potato, tomato, alfalfa, lettuce, maize, wheat, sorghum, rye, bananas, barley, oat, turf grass, forage grass, sugar cane, pea, field bean, rice, pine, poplar, apple, peaches, grape, strawberries, carrot, lettuce, cabbage, onion, citrus, cereal, nut plants, and other horticultural crops.

29. A method of providing a plant or plant part with a further desired agronomic trait comprising:
(a) inserting into the genome of plant material a polynucleotide which provides for the desired agronomic trait; and
(b) regenerating plants or plant parts from said material; and
(c) selecting the plants or plant parts having said desired agronomic trait wherein said plant material is capable of producing an insecticidal protein according to claim 1.

30. A method according to claim 29 wherein the further desired agronomic trait is selected from the group consisting of: herbicide resistance; insect resistance; nematode resistance; stress tolerance; altered yield; altered nutritional value or any other desirable agronomic trait.

31. Plants or plant parts obtained according to the method of claim 29.

32. An insecticidal protein consisting of the sequence depicted as:

| | |
|---|---|
| X₁-Gly₂-X₃-X₄-Cys₅-X₆-X₇-X₈-X₉-X₁₀-X₁₁-Cys₁₂-X₁₃-X₁₄-X₁₅-X₁₆-X₁₇-Cys₁₈- | (SEQ ID No. 34) |
| Cys₁₉-X₂₀-X₂₁-X₂₂-X₂₃-Cys₂₄-X₂₅-X₂₆-X₂₇-X₂₈-X₂₉-X₃₀-X₃₁-X₃₂-Cys₃₃-X₃₄- | |

wherein X_{1,3} and _{4, 6-11, 13-17, 20-23, 25-32} and ₃₄ is any amino acid.

33. The insecticidal protein according to claim 32 wherein X₁ is Glycine and X₃ is Lysine.

34. A method of controlling insects comprising providing at a locus where the insects feed a protein according to claim 1.

35. (canceled)

36. (canceled)

37. A recombinant micro-organism which produces a protein according claim 1.

38. A recombinant baculovirus which comprises a protein according to claim 1.

39. A method of controlling insects comprising exposing said insects to the recombinant baculovirus of claim 38.

40. An insecticidal protein which is capable of reacting with a monoclonal antibody raised to the protein selected from the group depicted as: SEQ ID Nos. 1 to 7.

41. A composition comprising an insecticidally effective amount of a protein according to claim 1 and optionally an agriculturally acceptable carrier and/or a diluent and/or an insect attractant.

42. A plant cell comprising the protein according to claim 1.

43. A polynucleotide encoding a protein according to claim 6.

44. A polynucleotide sequence which is the complement of one which hybridises to the polynucleotide according to claim 43 at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still encodes an insecticidal protein having a X-Glycine-(X-G-) motif at the N-terminus wherein X is any amino acid.

45. The polynucleotide sequence according to claim 43 comprising a sequence selected from the group depicted as SEQ ID Nos. 8 to 16, 37 and 38.

46. A synergistic insecticidal combination comprising a first protein according to claim 6 and at least one further insecticidal protein.

47. The combination according to claim 46 wherein said further insecticidal protein is a CRY protein.

48. The combination according to claim 47 wherein the said further insecticidal protein comprises a sequence selected from the group consisting of SEQ ID Nos. 27 to 32.

49. A polynucleotide which comprises regions encoding the first and further insecticidal protein according to claim 46.

50. The polynucleotide according to claim 49 wherein the region encoding said first protein comprises a sequence selected from the group depicted as SEQ ID Nos. 8 to 16, 37 and 38.

51. A DNA construct comprising in sequence a plant operable promoter operably linked to the polynucleotide according to claim 43 operably linked to a transcription termination region.

52. The DNA construct according to claim 51 which further comprises a region which provides for the targeting of the protein product to a particular location.

53. The DNA construct according to claim 51 which further comprises a region which provides for the production of a protein which acts as a selectable marker.

54. The DNA construct according to claim 51 wherein the plant operable promoter is selected from the group consisting of: PolyUbiquitin, Maize polyubiquitin, Rice pSS1, AoPR1, Actin2, Agrobacterium rhizogenes RolD; potato protease inhibitor II; CaMV35S; FMV35S; NOS; OCS; Patatin; E9; alcA/alcR switch; GST switch; RMS switch; oleosin; ribulose bisphosphate carboxylase-oxygenase small sub-unit promoter and other root specific promoters including MR7 promoter (maize); Gos 9 (rice), and GOS2 promoters.

55. A method of providing a plant or plant part with an insecticidal protein comprising:
(a) inserting into the genome of plant material a DNA construct according to claim 51; and
(b) regenerating plants or plant parts from said plant material; and
(d) selecting the plants or plant parts having said protein.

56. A method of providing a plant or plant part with an insecticidal protein synergistic combination comprising:
(a) inserting into the genome of plant material which produces an insecticidal protein, a DNA construct according to claim 51; and
(b) regenerating plants or plant parts form said plant material; and
(c) selecting the plants or plant parts having said combination.

57. A method of controlling insects comprising providing at a locus where the insects feed a protein according to claim 6.

58. A composition comprising an insecticidally effective amount of a protein according to claim 6 and optionally an agriculturally acceptable carrier and/or a diluent and/or an insect attractant.

59. A pant cell comprising the protein according to claim 6.
